(19) United States
(12) Patent Application Publication
(10) Pub. No.: US 2009/0221518 A1
(43) Pub. Date: Sep. 3, 2009

(54) Compositions and Methods for Treatment of Exudative Serous Effusion

(75) Inventors: Richard Kenley, Poway, CA (US); Mikhail Denissenko, Poway, CA (US)

Correspondence Address: DAVID P. LENTINI, 53 Clark Road, North Berwick, ME, 03906-6310, US

(73) Assignee: Anaborex, Inc., La Jolla, CA (US)

(21) Appl. No.: 12/205,183

(22) Filed: Sep. 5, 2008

Related U.S. Application Data
(60) Provisional application No. 60/988,802, filed on Nov. 18, 2007.

(51) Int. Cl. A61K 31/7048

(52) U.S. Cl. 514/29

(57) ABSTRACT

Compositions and methods are provided for treating accumulation of exudative serous effusions caused by illnesses such as cancer, infection, and pancreatitis. In one embodiment, the method of the invention comprises administering to such mammal a macrolide in an amount effective to prevent or at least alleviate said exudative serous effusion accumulation.

### 1 CROSS-REFERENCE TO RELATED APPLICATIONS

This application claims priority under 35 U.S.C. § 119(e) to provisional U.S. Patent Application Ser. No. 60/988,802, filed 18 Nov. 2007, the entire disclosure of which is incorporated herein by reference in its entirety and for all purposes.

### 2 BACKGROUND OF THE INVENTION

2.1 Field of the Invention

The present invention relates to compositions and methods for treating the clinical manifestations of exudative serous effusions. More specifically, the present invention relates to compositions and methods for treating exudative pleural serous effusions and exudative peritoneal serous effusions associated with various underlying pathologies such as cancer, infection, and pancreatitis. The invention has relevance to the fields of biology, medicine, oncology, and pharmacology.

2.2 The Related Art

Under physiological conditions, plasma permeates (effuses) continually from capillaries into the extra-vascular space to lubricate the serosal surfaces. Serous effusion can be classified as either transudative or exudative. Transudative serous effusion is defined by a process whereby portal pressure and plasma oncotic pressure control the effusion rate. Exudative serous effusion is the process whereby neovascularization and increased membrane permeability toward proteins control the effusion rate. Various methods have been devised to assist in the differential diagnosis of transudative versus exudative effusion-. Lymphatic drainage normally removes serous effusion and prevents fluid accumulation. When pathological conditions cause the effusion rate to exceed the lymphatic drainage rate, fluid accumulation occurs, often with serious clinical consequences.

Underlying diseases known to cause pleural effusion accumulation include: malignancy, congestive heart failure, tuberculosis, pneumonia, cirrhosis, and pulmonary embolism. Of these, congestive heart disease and cirrhosis cause transudative effusion, the other diseases cause exudative effusion. Similarly, underlying diseases known to cause peritoneal serous effusion (ascites) accumulation include: malignancy, congestive heart failure, tuberculosis, cirrhosis, and pancreatitis. The malignancies most often associated with exudative serous effusion are breast, lung, gastric, colo-rectal, ovarian, and pancreatic cancers.

When serous effusion accumulates in the space between the lungs and the pleural membrane, symptoms frequently include dyspnea (difficulty breathing), cough, chest pain, discomfort and anxiety. Accumulated serous fluid in the peritoneal cavity commonly causes pain, anorexia (loss of appetite), dyspnea, reduced mobility, and pseudo-obstructive symptoms. Together these symptoms contribute significantly to the suffering of and reduce the quality of life of afflicted individuals.

Transudate accumulation normally responds to treating the underlying condition (e.g., by reducing fluids in the body by administering diuretics). Existing therapeutic options for pleural and peritoneal exudative effusions, however, are more difficult and are often of limited effectiveness. For exudative effusions, surgical puncture and external drainage (centesis) is frequently performed on the pleural space (thoracentesis), or the peritoneal cavity (paracentesis), to remove accumulated fluid for diagnosis and symptom relief. Draining accumulated fluid provides temporary relief, but the fluid accumulation frequently reoccurs and serious complications of thoracentesis and paracentesis (such as bowel perforation and infection) are common. Other surgical and treatment approaches to exudative ascites also have limited effectiveness and serious drawbacks. Attempted pharmacologic interventions to prevent peritoneal and pleural effusion re-accumulation have met with little or no success.

The biochemical mechanism(s) controlling exudative serous effusion are still undetermined. Pro-inflammatory cytokines and the acute phase response may contribute to both benign and malignant cases, and neovascularization and increased vascular permeability to proteins are also factors in malignant pleural- and peritoneal serous effusions. With respect to cytokines and acute phase response, Alexandrakis, et al., determined concentrations of the pro-inflammatory cytokine, IL6, in serum and in accumulated serous effusion fluid taken from patients with malignant exudate, benign exudate, and transudate. Because IL-6 induces acute phase protein synthesis in the liver-, Alexandrakis, et al., also measured the C-reactive protein (CRP), a prominent acute phase protein. The authors concluded that IL-6 and CRP concentrations can distinguish exudative effusion from transudative effusion, and IL-6 concentrations in serous effusion distinguished malignant exudates from benign exudates. They postulated that tumor cells release IL-6 into the serous fluid that subsequently enters systemic circulation where it induces CRP formation. Other reports have also characterized cytokine and acute phase proteins in malignant and benign exudative serous effusions.

Macrolide antibiotics structurally related to erythromycin are known to decrease levels of pro-inflammatory cytokines and acute phase proteins-. Mikasa et al., demonstrated that treatment with the macrolide clarithromycin significantly increased median survival time for lung cancer patients and reduced IL6 levels. Another related macrolide, roxithromycin, has also demonstrated antiinflammatory effects, including the ability to reduce circulating levels of some pro-inflammatory cytokines-. Roxithromycin and related macrolides have been shown to limit exudate accumulation in rat paws injected with the potent inflammatory agent, carrageenan, when such macrolides are given to rats prior to carrageenan stimulation-. Roxithromycins mechanism of action is still unknown, but suppressing the nuclear transcription factor, NFB, may be an important factor.

Exudative serous effusion accumulation thus remains a frustrating and severe problem for clinicians and patients. Clearly, a medical need exists to simply, and effectively treat the accumulation of exudative effusions caused by diseases such as cancer, infection, and pancreatitis. The present invention addresses the treatment of exudative serous effusion and other needs.

### 3 SUMMARY OF EMBODIMENTS OF THE INVENTION

The present invention provides methods and compositions to prevent and treat the clinical manifestations of exudative serous effusion in mammals suffering from diseases such as cancer, infection, and pancreatitis. The methods and compositions of the invention are expected to improve the health of those suffering malignant and benign pleural and peritoneal exudative effusions that originate from a variety of disorders. The present invention also provides improvements of quality of life for patients suffering from such conditions in terms of: increased sense of well-being, enhanced appetite, reduced difficulty in breathing, and reduced pain.

In a first aspect, the present invention provides a method for alleviating the accumulation of exudative serous effusion in a subject. In a more particular embodiment, the macrolide is a macrolide antibiotic. In a still more particular embodiment, the macrolide comprises a 14 membered parent ring. In yet a more particular embodiment, the macrolide is roxithromycin, clarithromycin, or azithromycin, and, still more particularly, roxithromycin. In some of the latter embodiments, the roxithromycin is administered at a dose between about 25 milligrams per day (mg/d) and about 750 mg/d; a more specific embodiment is one in which the roxithromycin is administered at a dose between about 25 mg/d and about 300 mg/d.

In a second aspect, the invention provides methods in which, in addition to the foregoing administration, a paracentesis procedure is performed on the subject prior to the administration of the macrolide.

In a third aspect, the invention provides methods in which, in addition to the foregoing administration, a thoracentesis procedure of the subject prior to the administering the macrolide.

In a forth aspect, the exudative serous effusive condition is related to at least one of the following diseases: breast cancer, lung cancer, ovarian cancer, gastric cancer, colo-rectal cancer, or pancreatic cancer.

In a fifth aspect, the exudative serous effusive condition is related to at least one of the following diseases: tuberculosis or bacterial pneumonia.

In a sixth aspect, the exudative serous effusive condition is related to pancreatitis.

These and other aspects and advantages will become apparent when the Description below is read.

### 4 DETAILED DESCRIPTION OF SOME EMBODIMENTS OF THE INVENTION

In a first aspect, the present invention provides a method for alleviating the accumulation of exudative serous effusion in a subject. As used herein, alleviating the accumulation of exudative serous effusion in a subject means the reduction or elimination of exudative serous effusion in a subject suffering from such exudative serous effusion, regardless of cause.

In a more particular embodiment, the macrolide is a macrolide antibiotic, i.e., a macrolide possessing antibiotic properties as determined using methods known to persons having ordinary skill in the art. In a still more particular embodiment, the macrolide comprises a 14 membered parent ring, that is macrolide antibiotics having a central carbon ring structure such as shown below:

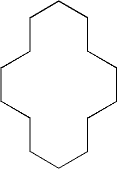

and includes various substituents at each carbon, as will be understood by persons having ordinary skill in the art. The identification of suitable 14 membered macrolide antibiotics will be apparent to those persons having ordinary skill in the art. In more particular embodiments, the macrolide is selected from the group consisting of: roxithromycin, clarithromycin, or azithromycin. In more specific embodiments, the macrolide is roxithromycin. In some of embodiments, roxithromycin is administered at a dose between about 25 milligrams per day (mg/d) and about 750 mg/d. In more specific embodiments roxithromycin is administered at a dose between about 25 mg/d and about 300 mg/d.

In other embodiments of the just recited method, the macrolide is administered in a pharmaceutically acceptable carrier. In more specific embodiments, the administration is by injection or oral delivery. In some embodiments, the administration is by oral ingestion at least once daily; in more specific embodiments, the administration is twice daily; in other embodiments, the administration is once daily. In some embodiments, the macrolide is injected into the peritoneal cavity of the subject. In other embodiments, the injection is made into the pleural or peritoneal cavity immediately following a thoracentesis or paracentesis procedure. The choice and preparation of oral and injectable pharmaceutical compositions suitable for use in the present invention will be known to those having ordinary skill in the art.

In still other embodiments, the above-described methods provided by the invention further include determining whether the exudative serous effusion condition is characterized by the presence of ascites in the peritoneal cavity of said subject. In other embodiments, the above-described methods provided by the invention further include determining whether said exudative serous effusion condition is characterized by the presence of accumulated fluid in the pleural space of said subject.

In still other embodiments, the exudative serous effusive condition is related to at least one of the following diseases: breast cancer, lung cancer, ovarian cancer, gastric cancer, colo-rectal cancer, or pancreatic cancer.

In other embodiments, the exudative serous effusive condition is related to at least one of the following diseases: tuberculosis or bacterial pneumonia.

In other embodiments, exudative serous effusive condition is related to pancreatitis.

The methods and compositions described herein are suitable for both humans and animals, especially animals of great economic or emotional value, such as, but not limited to: horses, cows, sheep, goats, pigs, cats, dogs, and the like, whether mature or immature (i.e., adults and children).

Patient progress can be determined using methods known to persons having ordinary skill in the art, such as, but not limited to, by measuring and observing relevant changes in the patients appearance (e.g., abdominal distention) or symptoms (pain, appetite, breathing ability). Patient progress can also be monitored using various imaging techniques such as X-ray, computerized tomography, and monograms. Progress can also be determined by: a reduction in the number of thoracentesis or paracentesis procedures performed over time, an increase in the time interval between thoracentesis/paracentesis procedures, and a reduction in the amount of accumulated fluid removed during each thoracentesis/paracentesis procedure. Patient progress can also be monitored by determining relevant clinical markers. Examples of such markers include, without limitation: levels of pro-inflammatory cytokines (IL-6 and tumor necrosis factor) and acute phase proteins (such as C-reactive protein) present in serum and in the accumulated serous effusion. The determination, measurement, and evaluation of such characteristics and markers associated with clinical progress are known to those having ordinary skill in the art.

In another aspect, the present invention also provides methods for preventing significant serous fluid accumulation in a mammal that is at risk of developing pleural or peritoneal exudative effusions because of an underlying pathology (cancer, infection, pancreatitis, etc). In these embodiments, any of the compositions and dosing regimens provided by the invention are administered to a mammal susceptible to developing exudative fluid accumulation to delay thereby the onset of fluid accumulation in an individual. The determination of any of these conditions can be determined using knowledge available to those having ordinary skill in the art, such as the knowledge of the underlying pathological condition, and/or determination of above-normal levels of biomarkers (e.g., IL-6, TNF, C-reactive protein).

Among the embodiments in which the macrolide is orally administered, the pharmaceutical carrier is a liquid (solution, syrup, emulsion, or suspension) suitable for oral ingestion or enteral administration using a naso-gastric tube. In other embodiments, the pharmaceutical carrier is a liquid (solution, suspension, or emulsion) suitable for injection of the macrolide into the peritoneal cavity or pleural space. In still other embodiments the pharmaceutical carrier is a solid (powder, sachet, tablet or capsule) dosage form suitable for oral ingestion that provides for immediate release of the macrolide into the gastric compartment. In other embodiments, the pharmaceutical carrier is a solid (powder, sachet, tablet or capsule) dosage form suitable for oral ingestion that provides for prolonged or controlled or sustained release of the macrolide into the gastric compartment.

In more particular embodiments, the pharmaceutical carrier for the oral liquid dosage form containing the macrolide is an aqueous solution of between about 5 millimolar (mM) to about 200 mM buffer (acetate, citrate, succinate, or phosphate) adjusted to a pH of between about 4 and about 7 containing about 3% to about 6% of a sugar such as sorbitol, sucrose, dextrose, lactose or mannitol plus between about 0.01% to about 1% of an antimicrobial preservative such as sodium benzoate or potassium sorbate plus various sweetening or flavoring ingredients known to those in the art and capable of dissolving the macrolide over the concentration range of about 0.5 milligram per milliliter (mg/mL) to about 10 mg/mL. In still more particular embodiments, the pharmaceutical carrier for the liquid oral dosage form containing the macrolide is an aqueous solution of about 10 mM to about 30 mM citrate buffer adjusted to pH between about 5.5 and about 6.5 containing about 4% to about 5% of mannitol plus about 0.05% to about 0.2% of potassium sorbate plus a sweetening or flavoring ingredient.

In more particular embodiments, the pharmaceutical carrier for the injection dosage form containing the macrolide is an aqueous solution of 5 mM to 200 mM buffer (acetate, citrate, succinate, phosphate) adjusted to pH between about 4 and about 7 containing about 3% to about 6% of a sugar such as sorbitol, sucrose, dextrose, lactose, or mannitol and capable of dissolving the macrolide over the concentration range about 0.5 mg/mL to about 10 mg/mL. In still more particular embodiments, the pharmaceutical carrier for the injectable solution dosage form containing the macrolide is an aqueous solution of about 10 mM to about 30 mM citrate buffer adjusted to pH between about 5.5 and about 6.5 containing about 4% to about 5% of mannitol.

In another particular embodiment, the pharmaceutical carrier for the solid oral dosage form containing the macrolide is a microparticle containing about 50% to about 90% of the macrolide co-extruded with micro-crystalline cellulose (MCC) plus a polymer binder such as hydroxypropyl methylcellulose (HPMC) that affords immediate release of the macrolide into the gastric contents. Immediate-release microparticles containing the macrolide may be filled into capsules or pressed into tablets using methods known to those skilled in the art. Tablets and capsules containing the immediate-release macrolide particles may also contain other inert, pharmaceutically acceptable excipients known to those skilled in the art, which may include, but are not limited to: lactose, starches, talc, or magnesium stearate.

In another particular embodiment, the pharmaceutical carrier for the solid oral dosage form containing the macrolide is a microparticle containing about 50% to about 90% of the macrolide co-extruded with MCC plus a polymer binder such as such as HPMC and overcoated with a polymer that affords sustained, or prolonged or controlled release of the macrolide into the gastric contents. Controlled- or sustained- or prolonged-release microparticles containing the macrolide may be filled into capsules or pressed into tablets using methods known to those skilled in the art. Tablets and capsules containing the sustained, or prolonged, or controlled-release macrolide particles may also contain other inert, pharmaceutically acceptable excipients known to those skilled in the art, which may include (but are not necessarily limited to): lactose, starches, talc, or magnesium stearate.

In all of the embodiments describing pharmaceutical carriers, the dosage forms may be prepared using methods and techniques known to those skilled in the art. Representative methods and techniques may include (but are not necessarily limited): mixing (blending, stirring, sonicating, levigating, emulsifying, homogenizing), grinding, milling, heating/cooling, filtering, filling (liquid or powder) coating (spray or fluidized bed), drying (air, heat, spray, fluidized bed, or vacuum), extrusion, spheronization, and compression.

### 5 EXAMPLES

The following Example of roxithromycin effects on exudative serous effusion (ascites) in a rat cancer model is provided to illustrate certain aspects of the present invention. The Example is in no way to be considered to limit the scope of the invention in any manner.

### 5.1 Experimental Details

Following the method of Busquets et al., female Wistar rats were inoculated (Day 0) intraperitoneally (ip) with 2×107 cells/animal of Yoshida AH-130 ascites hepatoma (AH) cells. Control animals were inoculated with an equivalent amount of sterile saline solution. Beginning on Day 1, the animals were given once-daily (qd) doses of roxithromycin by ip injection, twice daily (bid) doses of roxithromycin by oral gavage (po), or matching inactive vehicle. The roxithromycin dosing solution was 2 mg/mL in 20 mM citrate, pH of 5.5, and 4.5% mannitol in water.

The animals were sacrificed on Day 5. Upon sacrifice ascites fluid was withdrawn by syringe and the volume determined.

For the cell count measurements in ascites, the cells were counted directly from the ascites collected at necropsy from N 4 animals in Experiment 4. A 10 L aliquot was taken from each sample and diluted 1:500 with trypan blue. The sample was then placed onto a hemacytometer and both sides counted to determine the total number of viable and non-viable AH cells.

Serum samples were collected prior to sacrifice on D5 and assayed for serum albumin concentration. After determination of gastrocnemius muscle wet weight, some gastrocnemius samples were lysed and determinations made of C-reactive protein (CRP, using Quantikine Rat CRP Immunoassay Kit) and total protein. A commercial immunoassay kit (Quantikine Rat VEGF Immunoassay, R & D Systems PN RRV00) was used to determine Vascular Endothelial Growth Factor (VEGF) concentration in plasma and ascites. Similarly, CRP concentration in plasma and ascites was determined using a commercial kit (Rat High-Sensitive CRP ELISA Method, Kamiya Biochemical PN KT099).

### 5.2 Experimental Design

Three different experiments were performed. In each experiment one control group received an ascites hepatoma (AH) inoculation with vehicle treatment, and a second control group received saline inoculation with vehicle treatment.

In Experiment 1, the single treatment group received 5 mg/kg roxithromycin (ip). Ascites volumes were determined.

In Experiment 2, the single treatment group received 50 mg/kg roxithromycin (ip). Ascites volumes, serum albumin concentrations, and CRP levels in dissected gastrocnemius muscle were determined. AH cell counts and cell viability (%) were determined in recovered ascites fluid.

In Experiment 3, one treatment group received 40 mg/kg (ip) roxithromycin, and a second treatment group received 40 mg/kg (po). Ascites volumes, plasma CRP and VEGF concentrations, and ascites CRP and VEGF concentrations were determined.

Table 1 summarizes the experiments performed. Statistical significance was tested at the p 0.05 level.

TABLE 1
Experimental Design For Roxithromycin Experiments in Rat AH Model*

| | | | | | Ascites | | | | Plasma | | Serum | Muscle |
|---|---|---|---|---|---|---|---|---|---|---|---|---|
| Exp. | Group | Inoc. | Dose | | Vol. | AH | CRP | VEGF | CRP | VEGF | Alb. | CRP |
| 1 | 1 | AH | 0 | | + | − | − | − | − | − | − | − |
| | 2 | Saline | 0 | | N/A | N/A | N/A | N/A | N/A | N/A | N/A | N/A |
| | 3 | AH | 5 | (ip) | + | − | − | − | − | − | − | − |
| 2 | 4 | AH | 0 | | + | + | − | − | − | − | + | + |
| | 5 | Saline | 0 | | N/A | N/A | N/A | N/A | N/A | N/A | N/A | N/A |
| | 6 | AH | 50 | (ip) | + | + | − | − | − | − | + | + |
| 3 | 7 | AH | 0 | | + | − | + | + | + | + | − | − |
| | 8 | Saline | 0 | | N/A | N/A | N/A | N/A | N/A | N/A | N/A | N/A |
| | 9 | AH | 40 | (ip) | + | − | + | + | + | + | − | − |
| | 10 | AH | 40 | (oral) | + | − | + | + | + | + | − | − |

*A “+” sign indicates that the determinations were made for the indicated group. A “−” sign indicates that the determination were not made. “N/A” indicates “Not Applicable”, because no ascites accumulates in Saline control groups.

### 5.3 Results

Table 2 summarizes the data from Experiment 1. A 5 mg/kg (ip) dose of roxithromycin lowered the accumulated ascites fluid in rats inoculated with ascites hepatoma (AH) cells. The differences were not statistically significant, however.

TABLE 2
Ascites Volume Data for Rat AH Experiment 1

| | |
|---|---|
| Dose | Ascites (mL) |

| Group | Inoc. | (mg/kg) | Mean | SEM |
|---|---|---|---|---|
| 1 | AH | 0 | 4.04 | 0.67 |
| 2 | Saline | 0 | 0* | N/A |
| 3 | AH | 5 | 3.48** | 0.40 |

*The Group 2 versus Group 1 difference was statistically significant.
*The Group 3 versus Group 2 difference was statistically significant. The Group 3 vs Group 1 difference was not statistically significant.

Table 3 summarizes the Experiment 2 ascites data. In this experiment, 50 mg/kg roxithromycin very significantly reduced the accumulated ascites volume compared with the vehicle control group.

TABLE 3
Ascites Volume Data for Rat AH Experiment 2

| | |
|---|---|
| Dose | Ascites (mL)* |

| Group | Inoc. | (mg/kg) | Mean | SEM |
|---|---|---|---|---|
| 1 | AH | 0 | 6.05 | 0.78 |
| 2 | Saline | 0 | 0.00 | 0.00 |
| 3 | AH | 50 | 2.92 | 0.57 |

*The Group 6 vs Group 4 difference was statistically significant.

Table 4 shows the Experiment 2 data for serum albumin concentrations and CRP levels in gastrocnemius muscle. In vehicle-treated animals, serum albumin levels were significantly reduced in AH-inoculated versus saline-inoculated animals. The 50 mg/kg roxithromycin dose did not significantly change the serum albumin levels in AH-inoculated animals versus AH-inoculated animals receiving only vehicle. In vehicle-treated animals, muscle CRP levels were significantly increased in AH-inoculated versus saline-inoculated animals. The 50 mg/kg roxithromycin dose did significantly reduce the muscle CRP levels in AH-inoculated animals compared with AH-inoculated animals that received only vehicle.

TABLE 4
Serum Albumin and Muscle C-Reactive
Protein Data for Rat AH Experiment 2

| | | | Serum | | Muscle CRP | |
|---|---|---|---|---|---|---|
| | | Dose | Alb. (mL)* | | (ng/mg total protein)** | |
| Group | Inoc. | (mg/kg) | Mean | SEM | Mean | SEM |
| 4 | AH | 0 | 2.23 | 0.04 | 78.6 | 6.3 |
| 5 | Saline | 0 | 3.45 | 0.05 | 56.2 | 1.5 |
| 6 | AH | 50 | 2.36 | 0.12 | 28.5 | 0.96 |

*For Group 4 vs Group 5, and group 5 vs Group 6, the differences were statistically significant. The difference between Group 4 vs Group 6 was not statistically significant.
*For Group 4 vs Group 5, Group 4 vs Group 6, and group 5 vs Group 6, all differences were statistically significant.

Table 5 shows the ascites hepatoma cell density (cells/mL), cell count, and cell viability percent values for Experiment 2. IN AH-inoculated animals, the 50 mg/kg roxithromycin dose did not reduce AH cell densities or cell counts compared with the vehicle control. Furthermore, the AH cells were essentially all viable in roxithromycin versus vehicle control groups. Thus, the reduced ascites volume and reduced muscle CRP values in roxithromycin versus vehicle control groups cannot be due to cytostatic or cytotoxic activity of roxithromycin versus the AH cells.

TABLE 5
Cell Densities, Cell Totals, % Viable
Cells and for Rat AH Experiment 2†

| | Dose | Cells/mL × 10⁶ | | Total Cells # × 10⁶ | | Viable Cells % | |
|---|---|---|---|---|---|---|---|
| Group | (mg/kg) | Mean | STDEV | Mean | STDEV | Mean | STDEV |
| 4 | 0 | 113 | 25 | 1,330 | 405 | 96 | 0.90 |
| 6 | 50 | 338 | 23 | 2,077 | 1,007 | 99 | 0.60 |

†All groups were AH-inoculated. N = 4 animals/group.

Table 6 shows the Experiment 3 ascites volume and plasma CRP concentration data. In this experiment roxithromycin was given at 40 mg/kg by daily ip injection or by twice daily oral gavage. Plasma samples were tested for the concentration of vascular endothelial growth factor (VEGF), but plasma VEGF concentration values were all below the immunoassay method quantitation limit. In this experiment, 40 mg/kg roxithromycin given either po or ip very significantly reduced the accumulated ascites volume in AH-inoculated rats. In vehicle-treated animals, AH-inoculation lowered plasma CRP concentration versus saline-inoculation, but the differences were not statistically significant. In AH-inoculated rats, the 40 mg/kg roxithromycin ip dose lowered plasma CRP concentration compared with the vehicle-treated animals, but the differences were not statistically significant. Also in AH-inoculated rats, the 40 mg/kg oral roxithromycin dose very significantly lowered plasma CRP concentration compared with the vehicle-treated animals.

TABLE 6
Ascites Volume and Plasma [CRP] Data
for Rat AH Experiment 3†

| | | Dose | Ascites (mL)* | | Plasma [CRP] (μg/mL)** | |
|---|---|---|---|---|---|---|
| Group | Inoc. | (mg/kg) | Mean | SEM | Mean | SEM |
| 7 | AH | 0 | 6.7 | 0.7 | 732 | 45 |
| 8 | Saline | 0 | N/A | N/A | 838 | 31 |
| 9 | AH | 40, | 3.0 | 0.7 | 709 | 44 |
| | | i.p. qd | | | | |
| 10 | AH | 40, | 3.7 | 0.6 | 593 | 29 |
| | | oral, bid | | | | |

†Plasma VEGF concentrations were below the limit of quantitation of the immunoassay test method. The differences between Group 7 vs. Group 9, Group 7 vs. Group 10 were statistically significant. The differences between Group 9 vs. Group 10 were not statistically significant.
*The differences between Group 7 vs. Group 8, Group 9 vs. Group 8, and Group 10 vs. Group 8 were statistically significant. The difference between Group 10 vs Group 7 was statistically significant. The difference between Group 7 vs. Group 9 was not statistically significant.

Table 7 shows the Experiment 3 CRP and VEGF data for recovered ascites fluid. CRP and VEGF levels are expressed in concentration units (per mL of ascites fluid) and also as total mass of CRP and VEGF per animal (defined as concentration times volume of recovered ascites fluid). The CRP and VEGF concentration data were somewhat variable and none of the differences were statistically significant. The total VEGF per rat data also showed no statistically significant differences. For total CRP per rat, however, 40 mg/kg roxithromycin given either orally or intraperitoneally statistically significantly reduced total CRP values compared with AH-inoculated animals receiving only vehicle control. The total CRP values were not statistically significant between 40 mg/kg oral versus 40 mg/kg intraperitoneal roxithromycin.

TABLE 7
Ascites CRP and VEGF Levels in Rat AH Experiment 3

| | [CRP] | | CRP | | [VEGF] | | VEGF | |
|---|---|---|---|---|---|---|---|---|
| | (ng/mL)* | | ng/rat** | | (pg/mL)* | | pg/rat** | |
| Group | Mean | SEM | Mean | SEM | Mean | SEM | Mean | SEM |
| 7 | 285 | 33 | 1,410 | 270 | 2,570 | 300 | 12,100 | 2,400 |
| 9 | 230 | 52 | 530 | 250 | 4,590 | 1120 | 10,200 | 4,600 |
| 10 | 287 | 15 | 730 | 130 | 4,040 | 270 | 9,900 | 1,500 |

*None of the differences between groups were statistically significant at the p < 0.05 level.
*The differences between Group 7 vs Group 9 and Group 7 vs Group 10 were statistically significant. The difference between Group 9 vs Group 10 was not statistically significant.

### 5.4 Conclusions

The rat AH-130 tumor model has been extensively studied with respect to muscle wasting (cachexia)-, but surprisingly little attention has been afforded to the very significant (approximately 6 mL in a 100-gram animal) exudative serous effusion (ascites) accumulation that accompanies tumor growth in this model. Indeed, the dose-dependent activity of roxithromycin against ascites accumulation in this model, as demonstrated above, is a novel finding. Moreover, roxithromycin does not appear to be cytostatic or cytotoxic toward the ascites hepatoma cells. Thus, the evidence presented indicates that roxithromycin also does not influence VEGF levels, consistent with other reports regarding using macrolides in animal cancer models.

### 6 CONCLUSION

Thus, the methods provided by the present invention are effective in alleviating exudative serous effusion, and thus provide important adjuncts to treatment of many serious diseases, including cancers such as breast cancer, lung cancer, ovarian cancer, gastric cancer, colo-rectal cancer, or pancreatic cancer; tuberculosis or bacterial pneumonia; and pancreatitis. Although various specific embodiments and examples have been described herein, those having ordinary skill in the art will understand that many different implementations of the invention can be achieved without departing from the spirit or scope of this disclosure. For example, other antibiotic macrolides besides those described herein can be used with the methods described herein. Other methods of administration and formulation will also be apparent to persons having ordinary skill in the art. Still other variations will be clear to those having ordinary skill in the art.

What is claimed:

1. A method for alleviating the accumulation of exudative serous effusion in a subject, comprising administering to such subject a macrolide in an amount effective to at least partially reduce said exudative serous effusion in said subject.

2. The method of claim 1, wherein said macrolide is a macrolide antibiotic.

3. The method of claim 2, wherein said macrolide comprises a 14-membered parent ring.

4. The method of claim 3, wherein said macrolide is roxithromycin, clarithromycin, or azithromycin.

5. The method of claim 4, wherein said macrolide is roxithromycin.

6. The method of claim 5, wherein said roxithromycin is administered at a dose between about 25 mg/d and about 750 mg/d.

7. The method of claim 6, wherein said roxithromycin is administered at a dose between about 25 mg/d and about 300 mg/d.

8. The method of claim 1, further comprising administering said macrolide in a pharmaceutically acceptable carrier.

9. The method of claim 1, further comprising administering said macrolide by oral ingestion at least once daily.

10. The method of claim 9, further comprising administering said macrolide by oral ingestion twice daily.

11. The method of claim 9, further comprising administering said macrolide by oral ingestion once daily.

12. The method of claim 1 further comprising administering said macrolide by injection into the peritoneal cavity of said subject.

13. The method of claim 12, further comprising performing a paracentesis procedure on said subject prior to said administering said macrolide.

14. The method of claim 1 further comprising administering said macrolide by injection into the pleural space of said subject.

15. The method of claim 14, further comprising performing a thoracentesis procedure of said subject prior to said administering said macrolide.

16. The method of claim 1, further comprising determining whether said exudative serous effusion condition is characterized by the presence of ascites in the peritoneal cavity of said subject.

17. The method of claim 1, further comprising determining whether said exudative serous effusion condition is characterized by the presence of accumulated fluid in the pleural space of said subject.

18. The method of claim 1, wherein said exudative serous effusive condition is related to at least one of the following diseases: breast cancer, lung cancer, ovarian cancer, gastric cancer, colo-rectal cancer, or pancreatic cancer.

19. The method of claim 1, wherein said exudative serous effusive condition is related to at least one of the following diseases: tuberculosis or bacterial pneumonia

20. The method of claim 1, wherein said exudative serous effusive condition is related to pancreatitis.
